(19) United States
(12) Patent Application Publication
(10) Pub. No.: US 2024/0164675 A1
(43) Pub. Date: May 23, 2024

(54) METHOD FOR PREVENTIVELY DETERMINING A WELLBEING SCORE BASED ON BIOMETRIC DATA

(71) Applicant: Wellbeing.Ai BV, Vilvoorde (BE)

(72) Inventors: Nikolaos DIMITRIADIS, Vilvoorde (BE); Laurent VAN TORNHOUT, Vilvoorde (BE)

(21) Appl. No.: 18/516,910

(22) Filed: Nov. 21, 2023

(30) Foreign Application Priority Data
Nov. 22, 2022 (BE) 2022/5940

(51) Int. Cl. A61B 5/16; G06V 40/16; G06V 40/18; G10L 25/66; G16H 50/20

(52) U.S. Cl. CPC A61B 5/165; G06V 40/171; G06V 40/18; G10L 25/66; G16H 50/20; A61B 2562/02

(57) ABSTRACT

The present invention relates to an improved method for preventively determining a mental wellbeing score based on biometric data, specifically in relation to pre-emptively detecting burn-out and/or depression signs.

### CROSS-REFERENCE TO RELATED APPLICATIONS

This application claims priority to Belgian Patent Application 2022/5940 filed Nov. 22, 2022, the disclosure of which is hereby incorporated by reference.

### FIELD OF THE INVENTION

The invention relates to methods adopted for health monitoring of individuals. More specifically, the invention relates to systems and methods to measure behavioral health changes in individuals in different settings. The invention further provides for processing of acquired data by applying statistical, mathematical, and analytical tools to infer changes in the person's mental health.

### BACKGROUND

A number of psychological and physiological stress measurements exist, although no single gold standard metric for measuring stress exists. Heart rate variability (HRV) is the most commonly used measure of acute stress in academic literature as well as some commercial applications. Variability in heart rate occurs due to the opposing activities of the sympathetic and parasympathetic branches of the autonomous nervous system, which forms part of the SAM stress response pathway. However, it has been shown that HRV measurements are not a perfect representation of the sympathetic and parasympathetic systems and that these two systems are not correlated under all conditions. Research has shown that using HRV alone as a measurement of stress is an oversimplification of a complicated physiological process. Other measurements of stress that give an indication of SAM axis activation include heart rate, blood pressure monitoring, electrodermal activity measurement, respiratory rate measurement, and salivary a-amylase levels. Monitoring and quantification of stress levels is also achieved via assessment using psychological questionnaires in clinical conditions. All of these measurements are considered too simplistic or invasive and/or hard to measure and therefore do not offer reliable and/or continuous monitoring of stress levels.

Research studies have shown that cortisol levels can be reliably sampled from saliva and peak ten to thirty minutes after the induction of stress. The hormone cortisol is a key regulator of the stress response, and is synthesized from cholesterol in the adrenal glands. Blood cortisol levels peak ten to thirty minutes following a stressful event. Levels remain elevated for approximately one hour after the event. Elevated levels of blood cortisol activate a negative feedback loop system, which leads to a reduction of cortisol production causing blood cortisol levels to return to baseline. This negative feedback mechanism ensures that the body returns to homeostasis following a stress stimulus. Blood cortisol levels therefore serve as a biological marker of stress. Salivary cortisol has therefore become one of the most popular biomarkers for stress studies, and is the gold standard metric for activation of the HPA axis. A caveat of salivary cortisol measurements is that salivary cortisol measurements do not perfectly compare with blood cortisol levels due to the fact that some salivary cortisol levels are due to cortisone activity in the mouth and that samples require laboratory analysis. Therefore, it is difficult to obtain quick results or to enable continuous monitoring.

U.S. Pat. No. 8,622,901 describes a method for the continuous monitoring of stress in patients using accelerometer data combined with a number of other sensors including (but not limited to) heart-rate monitors, blood pressure monitors, pulse oximeters, and mood sensors. In order to enable continuous monitoring of stress levels using this method, a personalized stress profile is created for each individual patient from renal-Doppler sonography data, where the resistive index (R/I) of patients are used to calculate stress. A strong correlation exists between R/I and self-reported stress levels of patients. The relationship between R/I and self-reported stress levels are used to generate algorithms for calculating a stress index. The stress index is correlated with physiological and psychological data streams collected from the above-mentioned sensors, and a stress model for calculating the stress index as a function of physiological, psychological, behavioral, and environmental data is then determined. US20100022852 describes a method for processing galvanic skin response (GSR) signals to estimate the level of arousal of a user. GSR sensors measure the electrical resistance of the skin. Arousal of the sympathetic branch of the autonomous nervous system leads to an increase in sweat gland activity, which leads to an increase in skin conductance. Skin conductance can therefore be a measure of stress responses. The particular embodiment describes a computer program product for processing GSR signals which when run, controls a computer to estimate a level of arousal. EP2586365 describes a method for quantifying stress in a user, wherein the method allows for establishing discrimination between stressed users and relaxed users. The invention uses heart rate and GSR signals as data input, and utilizes stress patterns based on sigmoid transfer functions to allow quantifying stress in a larger number of situations. US20130281798 discloses methods for periodically monitoring the emotional state of a subject. Subjects are exposed to a plurality of stimuli during a session, wherein data is acquired through a plurality of physiological and psychological monitoring sensors. Data is transferred to a database, followed by data processing to extract objective information about the emotional state of a subject, specifically pertaining to emotional states—in clinical conditions—including, but not limited to, anxiety disorder, depression, mood disorder, attention deficit hyperactivity disorder, autism spectrum disorder, and bipolar disorder.

There remains a considerable need for biologically inspired systems and methods that can accurately, quickly, continuously, and non-invasively quantify and monitor an individual's stress levels, inside controlled or clinical environments, but also in more typical day-to-day settings. As described herein, systems and methods for accurate, continuous and non-invasive quantification of biological stress levels are disclosed.

Monitoring techniques of individuals with relation to mental health, have a limited set of objective clinical measurement tools available compared to general health practices, as well as relatively few scientifically well-grounded measurement tools in comparison to general health practices. Use of biometric measurement tools as measurement and monitoring devices is disclosed in several patents and patent applications.

U.S. Pat. No. 7,540,841 describes a system that collects data on an individual's daily activities to infer their mental health. U.S. Pat. No. 7,894,849 describes a method of collecting data through multiple sensors. WO2012108935 describes a health management system using a mobile communication device to communicate biometric sensor data through a server.

US 20130009993, US 20130011819, US 20130012790, and US 201300113331 disclose methodology to provide real-time feedback of health information for an employee from a set of health sensors, while the employee is engaged in work duties.

US20130281798 discloses methods and systems to periodically monitor the emotional state of a subject, comprising the steps of: exposing the subject to a plurality of stimuli during a session; acquiring objective data from a plurality of monitoring sensors, wherein at least one sensor measures a physiological parameter; transferring the data to a database; and processing the data to extract objective information about the emotional state of the subject.

US2020350057 and US2020302235 disclose methods to monitor biometric parameters to detect job-related mental health issues such as depression, by collecting biometric data and processing this via (potentially multilayered) machine learning models. However, it does not disclose a number of issues, such as computational optimization, guarantees on privacy of data, etc.

However, the above techniques generally require active cooperation of the individual, thus preventing them from performing ‘normally’ or doing their work, thus taking away key conditions that would regulate and determine their normal mental health situation. This in turn provides a bias to any results therefrom, and these can no longer qualify as objective measurements, given that they are no longer in the context of being determined during normal working conditions.

Another set of examples uses overly deep and strongly impactful neuroimaging methods of gathering data (electroencephalography or EEG for instance, and/or functional MRI or fMRI, and/or magneto-encephalography or MEG). While these provide more accurate readings of the mental health emotional states and neural correlates thereof typically, they are difficult to implement on a larger scale, are highly cumbersome to the individuals and remove the individual from their normal working conditions, while one of the main goals is to detect mental health in relation to said normal (and ecological, naturalistic) working conditions (amongst others), and the impact thereof on said mental health.

Finally, while there has been thorough research in detection of mental health issues based on such biometric data, this is typically performed by trained professionals, on an individual level, which is highly time-consuming and/or costly in terms of (external) hardware, and prone to human bias errors.

There is still a long felt and unmet need for improved methods and apparatus for treatment and monitoring of the mental health of individuals.

### SUMMARY OF THE INVENTION

The invention relates to an improved method of assessing an individual's mental state, in particular with relation to preventive detection of depression and/or burnout. The method uses easily collectable biometric data, for which analysis and modelling of the data also translates well into strong correlations that can be drawn to mental state of an individual. Burnout has been confirmed by the World Health Organization (WHO) as a symptom of chronic, non-treated, work-related stress, which was accentuated during and after the COVID-19 pandemic.

One of the first requirements in the method is that it can used during normal working conditions, i.e., that there is no substantial physical and/or mental impact during the acquisition/collection phase of the biometric data on the individual. This means that neuroimaging methods, such as electroencephalograms, are not suitable, as are galvanic skin response and/or high-precision infrared technology measurements in most cases. This restriction is strongly based on the fact that the objective is to see the impact of the work conditions (mental and/or physical) on the subject, without a bias due to the strong feeling they are being monitored. Preferably, the acquisition is performed while the subject is not aware of this, and is thus not limited or bothered by the data collection (having approved the acquisition, but not being made aware of exact timing thereof). This creates a situation where the subject behaves naturally and does not tint the readings.

One method that is particularly suitable for this restriction, is the use of audiovisual information. This can be gathered easily and quasi undetectably, for instance via a webcam on the workstation (computer, laptop or similar) or an integrated external camera. Audiovisual information provides a treasure trove of information on the individual's behavioral correlates of the emotional state, that can be detected by using techniques such as eye tracking data (“they eyes are the window to the soul”), facial expression analysis data and voice analysis. These techniques/technologies are especially suitable in settings where monitoring should not impact ongoing tasks and performance. This data provides very reliable insights, in large volumes and with high time frame granularity.

A further desired feature is that the method allows monitoring of the individuals over prolonged periods of time, while they can keep performing their tasks, to a large extent at least. This avoids the monitoring to impact the performance, which could again bias the readings, as it creates anxiety for not reaching certain goals or meeting deadlines.

A further requirement is anonymity for the individual, while retaining the possibility to track data back to them for further analysis, in order to combine data from an individual, taken at different points in time and/or via different intake devices and data capturing solutions, to arrive at a conclusion. This requires a carefully set up data structure, ensuring that there a traceability for data and individuals without risking that information and link to become publicly available to unauthorized users.

A final feature that is highly sought after, is an objective, unbiased reading. While human operators could peruse the data, however time-consuming and expensive this would be, and provide evaluations based thereon, it will almost inevitably be under a certain bias, as this data is almost impossible to anonymize, since it would render the data irrelevant for analysis. As such, human evaluation is avoided entirely (except perhaps for quality control and review of anomalous results), and a number of predictive, preferably machine learning, models are applied to the data stream, and constantly trained with said data.

As such, the invention comprises the following steps:
a) Acquiring audio-visual information via an electronic device provided with an image sensor from each of the individuals during work operations of each of the individuals over prolonged periods of time, said audio-visual information comprising video and/or images and associated audio output;
b) Acquiring biometric data for each of the individuals from said audio-visual information, wherein said audio-visual information is processed into the biometric data by a processor in said electronic device, said biometric data comprising at least:
   eye tracking data, comprising at least eye position and eye movement for each of the individuals, acquired from video and/or images;
   facial expression analysis data, acquired from said video and/or images;
   voice analysis data, acquired from said audio associated output;
c) Processing said biometric data for each of the individuals, said processing comprising the following substeps, wherein the biometric data is processed by the processor in said electronic device:
   a. Performing a feature extraction algorithm on the biometric data, resulting in an extracted feature set;
   b. Performing a feature selection algorithm on the extracted feature set, resulting in a reduced feature set;
d) Feeding the reduced feature set for said biometric data, preferably after step ii. of the processing step and a behavioral data set to a first predictive model, said behavioral data set comprising data associated to each of the individuals, said first predictive model and comprising at least physical health information and/or mental health information, preferably using a first machine learning model, and said first predictive model defining values for a predefined first set of key performance indicators (KPIs) from the features of the reduced feature set, wherein said first predictive model is executed on the electronic device;
e) Sending the first set of KPIs to a remote server, preferably a cloud server;
f) Feeding the first set of KPIs, and the behavioral data set to a second predictive model at said remote server, thereby defining values for a predefined second set of KPIs, said second predictive model preferably using a second machine learning model, wherein said second predictive model is executed at the remote server;
g) Feeding the second set of KPIs, and optionally the behavioral data set to a third predictive model at said remote server, thereby defining values for a predefined third set of KPIs, said third predictive model preferably using a third machine learning model, wherein said third predictive model is executed at the remote server, said third set of KPIs preferably relating to a single KPI;
h) Determining a mental health assessment for each of the individuals separately based on said values of said third set of KPIs.

While it is known that voice, eye tracking and facial expression analysis can reveal much about an individual's state of mind, the present state of the art remains silent on using this combined data to feed and train a predictive model that can for instance operate via machine learning. Equally important in this context, is that the first (and optionally second and third) model is also fed with behavioral data from the individuals. Said behavioral data can provide a necessary context for the biometric data that the system pulls from the individual, which is often missing and provides for faulty or needlessly alarming results, as well as accounting for algorithmic bias.

By defining sets of KPIs at each level, parameters are predefined of which clinical and experimental data have shown the relevance towards the wellbeing of an individual. The first set of KPIs are such parameters which are more clearly derivable from the biometric data, with each further set building onto the previous layers and sets, optionally along with (part of) the original biometric data. The parameters can be for instance quantifications of a level of anxiety, burnout, engagement, satisfaction, immersion, confrontation, etc.

The second (and third) set of KPIs typically builds on top of two or more of the KPIs of the underlying first (and/or second) set of KPIs, which two or more KPIs have some resonance with each other. For instance, engagement and satisfaction may result in a quantification for enjoyment, etc.

Further information on the KPIs of each set is provided further on in the form of a possible embodiment.

Behavioral data can comprise one or more of the following information:
Physical health information (pregnancy, illness, physical problems, . . . )
Mental health information (stress, anxiety, home situation, loss of a person close to the individual, . . . )
Work information: holidays, shifts, . . .
Other information

Preferably, this information is provided via the individual themselves and/or via colleagues (for instance HR data). Some of this information can provide context for abnormal readings, such as due to pregnancy, first day after prolonged holiday, etc., and can avoid skewed readings from divergent biometric data. For instance, perceived stress levels and the associated biometric readings directly after or before a prolonged holiday may strongly differ from ‘normal’ stress levels. Similarly, a pregnancy, loss of a loved one, etc., can strongly affect the emotional condition of the individual, and often leads to non-representative data which could be quite alarming.

The information can be gathered periodically, via a questionnaire for instance.

The information can be gathered via multiple-choice questions and/or open questions, which can be converted via Natural Language Processing (NLP) into processable input for the predictive models.

Preferably, the behavioral data serves as context information for the biometric data, and is not used directly. The context information can serve to discard certain sections of biometric data, or to provide a reduced weight for such sections.

By using the behavioral data, the predictive models can learn how to assess weighted biometric data (influenced by personal affectations of the individual), and correctly evaluate the mental state of the individual.

Furthermore, by providing separate predictive models, who are in turn fed by each other's results (and optionally behavioral data), a more objective approach is formulated, and a higher accuracy is achieved in assessing the mental state.

The methods known in the state of the art remains silent on most, if not all, of the points raised above.

### DESCRIPTION OF FIGURES

FIG. 1 shows an overall process flow for an embodiment of the invention, supplemented with a number of optional techniques.

### DETAILED DESCRIPTION OF THE INVENTION

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

In the present document, reference is made to “mental health” and “mental state”. It should be understood that such terms also cover, and can even present a direct equivalence to, “emotional health” and “emotional state”, which are the terms more commonly used in the field of psychology, with the former being medical terms.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

The invention relates to a computer-implemented method according to claim 1.

By determining values for predefined KPIs of the first set from the original biometric data set (typically after feature extraction and feature selection algorithms being performed on the biometric data set) with a first predictive model, these first findings can be fed to a second predictive model that builds further on the learnings of the first model to determine values for predefined KPIs from the second set, which in turn feeds a third model to determine values for predefined KPIs from the third set. This reduces the risk of bias in the results, and increases the chances that anomalous readings are detected and managed.

Furthermore, the method provides for a separation of the computational load over the electronic device at the user station, and a remote server, implementing an edge computing approach to the system's architecture. The audio-visual information is processed, first into biometric data, and then into values for the first set of KPIs, on the electronic device of the user. The resulting KPIs and values therefor are then sent to the remote server, at which the second and third predictive models process the data further. The above provides for the added advantages of privacy and data security. Biometric data itself is not sent from the user device, thus securing this data from unwanted interference and viewing. Additionally, a much lower volume of data needs to be transferred, as the data-heavy video and audio information is processed locally into values for a number of KPIs (and can optionally be deleted automatically). Instead of continuously forwarding an audio/video feed to a remote server, only a few values are forwarded at periodical intervals, for instance during set sample periods of at least 20, preferably at least 50 ms, more preferably at least 100 ms, or for instance 200 ms.

Another advantage is the low latency in the process. Using edge devices (for instance computer of the individual at the workstation) that acquire the audio-visual information (or receive it from an external device) allows a first processing step that deals with high volumes of data, to be performed locally, freeing up the remote server from this job, and creating a parallel computing system in which all the local edge devices perform this first step. The remote server thus only needs to work on a significantly reduced data set, namely the first set of KPIs, and optionally behavioral data.

The first predictive model essentially starts from an unlabeled data set, and attempts to label these, based on rudimental insights from the behavioral data to guide it. From this first analysis, a set of KPIs is distilled, which serve as additional input for the next predictive model, and is again supplied with the biometric data and optionally the behavioral data, a process which is repeated for the KPIs from the second predictive model.

It should be noted that especially the above features and advantages are wholly undisclosed in prior art, for instance in US2020350057 and US2020302235. In the known methodologies, full data sets are forwarded from the edge devices to a central server, where the full processing takes place. Not only is this a strongly suboptimal distribution of computational load (single point for all processing), but it furthermore requires a total transfer of all collected data from the edge devices to the central server, which taxes the systems unnecessarily, wastes bandwidth and can jeopardize the highly confidential data in the transferred data as it provides the potential for security breaches. In the present invention, the collected biometric data is processed locally, using the processing power of the edge devices for a first processing step as well deriving the first set of KPIs. This strongly or even entirely anonymizes the data but also immensely reduces the volume thereof that is then actually sent to the central server for further processing.

The step of feature extraction starts from the full set of biometric data and converts this to derived features for the data, which is representative thereof but phrased in a way that allows better processing in subsequent (machine learning) step. A high number of feature extraction techniques are known, such as PCA (Principle Component Analysis), Kernel PCA, Factor Analysis, ICA (Independent Component Analysis), LDA (Linear Discriminant Analysis), LLE (Locally Linear Embedding), t-SNE (t-distributed Stochastic Neighbor Embedding), Autoencoders (with such variations as denoising, variation, convolutional, sparse, etc.), and/or others. This results in (mostly) new features, though of course in some cases, where relevant features are directly present in the biometric data set, these will be retained.

Features to be extracted can be anything that is relevant for the data set, and represent individual measurable properties or characteristics for the data set, such as statistical parameters (mean, minimum, maximum, median, average, variance, etc.), function parameters and others.

Features in the present application may also relate to information retrieved from (relevant image) and/or audio samples (typically said samples having particular relevance/trustworthiness and/or being unambiguous), and the identification of certain parameters in said image/audio samples, such as for instance recognition of emotion in face/eye/voice, eye-gaze patterns, movement of facial features, etc., as well as the intensity thereof, length thereof, etc. By identifying and quantifying these features, the predictive models can develop values for the KPIs.

Based on these features, values are determined for the first set of KPIs, by the first predictive model. This can be via fixed mathematical formulas, where the features are variables, or via variable formulas which are adapted over the course of training/improving the models (for instance automatically by Machine Learning or manually by new insights).

Preferably, the audio-visual information and biometric data is analyzed in segments, of a predefined length, for instance at least 20 ms, or 50 ms, or 100 ms or more, and at most 1000 ms, or at most 500 ms or 250 ms. From each of these segments or samples, the value for the first set of KPIs is determined for said segment/sample, by detecting certain indicators associated to a KPI. This can be based on eye-movement speed, direction, voice fluctuations, and other of the biometric data, or combinations thereof.

Such a value can for instance be a score indicating the likeliness of the presence of a certain KPI in the segment/sample (e.g., 1-100). This score does not necessarily need to be linear or continuous, although preferably it is, but can function according to certain thresholds.

Note that some KPIs can receive values by combining multiple segments, or even all segments, as they require longer observation.

These values are then forwarded to serve as input for determination of values for the second set of KPIs, etc.

In a particularly preferred embodiment, the first set of KPIs comprises one or more of the following (and preferably all):
Positive valence: Intensity and probability for positive emotions to be present; score preferably determined by detection of reaction of the individual to the screen (or other working tool) and presence of a significant (positive) variance from a baseline set at the beginning of the session.
Interest: Sustained attention an individual can keep while scanning a screen, representing mental absorption in a task; score preferably determined by calculation of probability of an individual's interest present when scanning patterns related with interest are maintained.
Focus: Level of vigor and persistence with what an individual is involved in a task; score preferably determined by calculation of probability of an individual's focus present when scanning patterns related with fixated attention in specific points in the visual field.
Confidence: Sustained approach behavior which occurs partially as the result of specific pleasurable sensations and seeking behavior; preferably determined by calculating the probability of an individual's confidence present when scanning patterns and facial movements related with the combination of pleasure, gaze seeking and need satisfaction.
Negative valence: Intensity and probability for negative emotions to be present; score preferably determined by detection of reaction of the individual to the screen (or other working tool) and presence of a significant (negative) variance from a baseline set at the beginning of the session.
Stress: Relationship between the person and the environment that is appraised as personally significant and as taxing or exceeding resources for coping; score preferably determined by calculating the probability of an individual's stress present when altered fixation behaviors that do not follow a coherent scanning pattern. Longer fixation on objects related to a specific area. Also, diminished face fixation suggests gaze avoidance of aversion to a certain stimuli on screen.
Avoidance: Adaptive mechanism to flight ad leave conflicts and unpleasant experiences; score preferably determined by calculating the probability of an individual's avoidance present when a focalized scanning pattern is interrupted by a distant look away or a disengagement pattern, as well as fear and disgust indicators.
Performance: Level of effectiveness in which an individual completes a task; score preferably determined by calculating the probability of an individual's performance present when scanning patterns are assertive and congruent.

It should be noted that these values can shift strongly from segment to segment and are snapshots often without much further meaning when considered in isolation. This means that the values often are hard to evaluate, especially when we take into account that such snapshot values may be incorrect. A goal of the system and methodology is to remove identifiably wrong values, but there is no guarantee that ‘small’ mistakes are filtered out. It is important to process the resulting values into broader terms that provide a better handhold for (intuitive) understanding, as they are combined with other values that can balance each other. Even then, such terms may still present values for a ‘parameter’ over too short of a timespan to have much significance. It is in this light that the values for KPIs (especially at the second and third set of KPIs) can be averaged over longer periods of time, presenting a better view on the individual's state of mind.

It is to be understood that in the calculation of values for higher-level KPIs (for instance, going from KPIs from set 1 to KPIs of set 2), the values of the underlying KPIs can be implemented differently. For instance, for determining the value of a first KPI of the second set, the value of a first KPI of the first set is used in its direct form (namely, the direct value), while for the value of a second KPI of the second set, the first KPI of the first set is also used, but here an average is used of said value of the first KPI of the first set over a certain amount of time. Both the first and second KPI of the second set use the same KPI of the first set, but in a different manner, which can produce a strongly different result. Other operations may also be performed on the values of the underlying KPI sets before use in setting the value of upper KPIs, such as logarithm of the value, a power of the value, or any other function that can be applied to the value. It can also be combined with one or more other KPIs in such a function (for instance KPI 1×KPI 2, etc.).

As is clear from the above, the only data that is sent are values for the KPIs (no audio-visual information or biometric data), which can easily be processed further at server-side, with low latency. This allows the methodology to work at practically any number of users at the same time, as the computational load is mainly borne on user-side work stations, and is thus well-distributed.

The second set of KPIs is scored in value based on the values for the first set of KPIs. The way in which the scoring is performed may vary over time. Preferably, each of the second set of KPIs is based on a subset (potentially overlapping with other subsets) of the first set of KPI values.

For instance, one of the second set of KPIs may be determined based on the value of positive valence, interest and focus. Another may be determined based on the value of negative valence and stress, while still another may be based on positive valence (again) and confidence, with another based on negative valence (again), avoidance and performance.

In a particularly preferred embodiment, the second set of KPIs comprises one or more of the following (and preferably all):
Engagement: Positive, fulfilling work-related state of mind that is characterized by vigor, dedication and absorption; scored preferably based on positive valence, interest and focus.
Joy: Pleasurable experience associated to a specific task; scored preferably based on positive valence and confidence.
Anxiety: Diffused emotional state caused by a potentially harmful situation, with the probability or occurrence of harm being low or uncertain; scored preferably based on negative valence and stress.
Burnout: State of exhaustion and cynicism towards work; scored preferably based on negative valence, avoidance and performance.

The way of determining the value for the second set of KPIs from the values of the underlying KPI set is typically via a predetermined formula.

Again, we note that the determined values are difficult to treat or understand when only considered for a single point in time, and especially in isolation from each other. The KPIs provide context and balance for each other, but preferably, the score should be considered averaged over a certain amount of time. Typically, this is over the length of the entire session, but on some occasions, the session can (simultaneously) be evaluated in subsections, especially when the metrics for certain subsections are worrying, and this can then be used to flag underlying issues which may not be visible from the overall values determined.

When the term “averaging” or “averaged” is mentioned, this can refer to a number of statistical mathematical techniques, such as arithmetic means, median, geometric median, Turkey median, mode, geometric mean, harmonic mean, Lehmer mean, quadratic mean, cubic mean, generalized mean, weighted mean, truncated mean, interquartile mean, midrange, Winsorized mean, etc. More particular, customized averages can also be employed, where for instance values over/under a certain threshold are given more or less weight than others, are discarded, etc.

The averaging technique applied can be chosen to differ for each KPIs.

Most preferably, the third set of KPIs relates to a single value, a supposed “wellbeing score”, which takes into consideration the values of the KPIs of the second set (in the above example: “engagement”, “joy”, “anxiety” and “burnout”).

As mentioned above, the determined value is preferably averaged over a prolonged period of time (e.g., entire session), but can be studied over shorter timespans to identify troublesome situations which may not appear from an averaged result.

The step of feature selection departs from the set of features resulting from the feature extraction step, and aims to further reduce the feature set by removing redundant and/or irrelevant features therefrom, and results in a reduced feature set, that is a subset of the original feature set before selection (this as opposed to feature extraction, where new features are generated in terms of functions of the original data). This makes the data set more manageable for further analysis, and aims to avoid that irrelevant data impacts the final analysis too strongly or too directly (for instance, by having multiple features on essentially the data). Known feature selection methods typically fall in a few groups: wrapper methods, filter methods and embedded methods.

The first step of feeding the biometric data and feature sets derived therefrom, as well as the behavioral set, to a first predictive model (preferably operating according to a machine learning model), allows the method to provide a first, point blank evaluation on KPIs (key performance indicators) in the biometric data. This regulates the further steps in part, by providing features which are likely very relevant in the diagnosis.

By feeding the newly defined KPIs (optionally with the behavioral data) from the previous predictive model, into a subsequent predictive model, these subsequent models are guided by their predecessor, to avoid that the final result is biased, or functions overall as a black box. Having multiple subsequent models analyze the data reduces the risk that wrong conclusions are drawn from biases in one model.

In some embodiments, the data and associated features are analyzed by cluster analysis in the models. Cluster analysis is the act of grouping a set of objects in such a way that objects in the same group (called a cluster) are more similar to each other than to those in other clusters. An example of cluster analysis is k-means clustering, which is used to classify measurements, the biometric data, derived from one or more data acquisition devices (webcam in many cases) into different stress and activity level states. Another example of cluster analysis is fuzzy clustering implemented by fuzzy clustering analysis module. Fuzzy logic is a form of computer logic, the output of which is a continuum of values between 0 and 1 which can also be represented as 0-100%. The system starts by assigning a set of membership functions for each input and a set for each output. A set of rules for the membership function is then applied. In particular embodiments, the algorithm allows the k-means clusters to inform the shape of the membership function. Fuzzy clustering provides an indication of the percentage of which of the features in the data belong to a particular cluster or state. It is therefore possible to determine the level of stress (from 0-100%) that a subject is experiencing. The output of fuzzy clustering is a stress function that fluctuates with time as stress levels rise and fall. A Gaussian mixture model (GMM) module is another example of clustering model. The GMM model assumes that all the data points are generated from a finite number of Gaussian distributions with unknown parameters. In particular embodiments, the GMM model offers an advantage by combining the clustering process and the stress function calculation in one model. In particular embodiments, a stress rotation model module is used for the classification of acute and chronic stress and exercise events. Vector directionality of data points on a parametric plot may be visualized as loops or “rotations”. Rotational measurements may correspond to stress or exercise events. In order to capture and quantify the information generated by rotations, an algorithm calculates the area of rotations for both stress and exercise. The area is output as a stress function indicating the duration and severity of the stress or exercise event. This model offers an advantage over clustering techniques in that it has the ability to predict both acute mental stress and exercise at the same time. In other embodiments, stress intensities are calculated by using a combination of K-means clustering, fuzzy clustering GMM, and stress rotation algorithms. The above-mentioned theory and methods are used to create an ensemble to classify stress events. Stress intensities are subsequently calculated using, as an example, but not limited to, logistic regression functions using a minimum of one biometric data parameter as input to guide the intensity.

In the above, mention is made of stress intensity, but of course, this is only a general name for what is determined via the present method. In many cases, stress intensity will be one of the relevant parameters for a final diagnosis.

It should be noted that the methodology described above for stress intensity can be applied for the determination of other parameters.

In some embodiments, further biometric data sets are used in the analysis, for instance breathing pattern data, which can also be acquired derived from audio-visual information (webcam for instance).

Alternative or additional data sets can be temperature data (acquirable via IR data for instance); motion data (visual information) which can show twitching, nerves, physical problems, etc.; and/or others.

Further biometric information can be comprised in the biometric data set as well, such as mouse movement data, which has been proven to show indications of an individual's mental state (speed, movement patterns, oscillations, reaction time, etc.).

Other additional biometric information can for instance comprise information regarding blood vasculature (determinable from video and/or images, and/or specialized equipment), basic ECG readings via wearables such as earplugs, etc.

In a preferred embodiment, the biometric data is processed with a first substep of pre-processing the biometric data. The pre-processing comprises at least denoising of at least part of the biometric data (preferably all of it). Preferably at least the audio output is denoised as this is typically most riddled with noise, making voice analysis very difficult. The step of pre-processing the biometric data precedes the substep of performing a feature extraction algorithm on the biometric data.

In a further preferred embodiment, the steps of pre-processing and feature extraction of the biometric data is performed per set of eye tracking date, facial expression analysis data and voice analysis data separately for each of said sets.

In a preferred embodiment, the step of processing the biometric data comprises a substep of automatedly detecting anomalous data and/or features in the biometric data, in the extracted feature set and/or in the reduced feature set, and subsequently automatedly removing and/or automatedly adapting anomalous data and/or features therefrom. Where many of the prior art methodologies fail, is that anomalous data is improperly filtered, adjusted or removed when processing. Such values inevitably skew the result, and as the methodology is specifically aimed at rapid detection of future mental health issues, strongly divergent values will generally lead to a strong diagnosis, i.e., a warning that the individual is facing substantial issues, under severe stress, etc. In order to avoid these false positives, the present techniques strongly analyzes the anomalous values, and removes or adapts these before proceeding to a next step.

In general data analysis, such extreme values are often maintained as pointing to severe problems. However, in the understanding that the data relates to biometrics, extreme values are highly unlikely (e.g., voice analysis data pointing at frequency ranges of over 50 kHz, which is much higher than the maximal range for a human voice, should point at mechanical failure, and therefore removed from readings).

In a preferred embodiment, at least two, and preferably each, of the first, second and third predictive models are different predictive models, said predictive models preferably being machine learning models (which are different machine learning models). By using different ML models, bias is reduced, and the results will be more relevant and reliable.

The ML models can operate under supervised learning, unsupervised learning, semi-supervised learning or reinforcement learning.

The ML models can follow algorithms chosen from the following list, but are not limited thereto: linear regression, logistic regression, decision tree, SVM algorithm, Naïve Bayes algorithm, KNN algorithm, K-means, Random forest algorithm, dimensionality reduction algorithm, gradient boosting algorithm, AdaBoosting algorithm, and/or others.

Preferably, the first, second and/or third ML models are selected from: Random forest, Support Vector Machine (SVM), Relevance Vector Machine (RVM), Perceptron, Artificial Neural Network (ANN), K-Means Clustering, k-Nearest Neighbours (k-NN).

In a preferred embodiment, the biometric data is, preferably exclusively, collected via a webcam (and preferably an inbuilt or separate microphone device) at a working station of each of the individuals. Under this restriction, it is understood that in case of a separate audio intake device (microphone) in the computer or laptop or external thereto at the working station is considered to form part of the webcam, as would be for instance an inbuilt camera in the computer or laptop at said working station.

Advantages thereof lie in the fact that it allows an easy and qualitative data collection procedure, without a substantial burden to the individual from which the data is collected.

In a preferred embodiment, the prolonged periods of time span at least 10 minutes, more preferably at least 30 minutes, wherein preferably multiple sets of audio-visual information are acquired over said prolonged periods of time for each of the individuals. By having multiple data sets, the reliability and accuracy are increased. The same applies for lengthier data acquisition sessions. It is likely that the initial phase of each period is riddled with anomalous, for instance due to nerves of the individual, which wane after a few minutes. By having the period last over at least 10 minutes, this ensures that at least part of the data set provides relatively objective and untampered data points. In some embodiments, it can be envisioned that a first part of the data is removed from further processing, for instance the first minute, 2 minutes, 3 minutes, etc. Alternatively, this cutoff can be defined by comparison of the data over the entire period. If a baseline can be detected in the dataset, then the initial period in which the data deviates from the baseline over a predefined threshold, can be wholly or partly removed. In some variations, this data set can however be used separately, as it can provide insight on the nervousness, stress level or other emotional states of the individual, and can provide further features of interest for diagnostic purposes.

In a preferred embodiment, the individuals are divided in groups based on work content of the individuals, wherein group average comparison values are determined by averaging the biometric data of the reduced featured set based on the third set of KPIs for each of the individuals in a group, and wherein the step of determining the mental health assessment for each of the individuals separately further takes into account the group average comparison values for the group to which the individual belongs.

It is found that different jobs provide strongly different influences on the individual's mental state. While it is interesting to use the retrieved data, and processed results, from individuals to compare, this is therefore often troublesome, as averaged results often balance each other out, or are skewed by the larger groups (and their results). By grouping the individuals based on job description (work content), it is found that a group-based comparison is more relevant and provides more insights.

In a preferred embodiment, the predictive models are furthermore fed with situational data associated to each of the individuals, said situation data comprising at least a geographical denomination to the work environment of the individual. Such situational data can comprise geographical location, information regarding time zone of work, but also more specific work information (work in shifts, size of teams, incidents at work, etc.). On many occasions, mental health knows shifts depending on location, while the mentioned other influences can impact this as well. By taking this into account, anomalous data can be recognized more efficiently, but further or different groupings can be made (see above), for average comparison values to arrive at a diagnosis.

In a preferred embodiment, the biometric data is supplemented with a task description for the work operation of the individual during the prolonged period of time, said task description preferably selected from a predefined list of task descriptions. Type of work also impacts mental health, but can also strongly factor into the biometric readings. An individual working an office job in front of a computer provides starkly different results than an operator at a conveyor belt. Again, this can be taken into account for anomaly detection and elimination, as well as grouping.

In a specific embodiment, this allows the method to determine task-associated values for the KPIs (first, second and/or third set), in order to determine a mental health assessment for the individual per task. By doing so, specific actions can be undertaken to improve user wellbeing as well as efficiency for specific tasks, for instance by instituting working from home policies for certain task (expressly stimulating or disallowing it for certain tasks for instance). This can be improved even further by taking into account additional situational information regarding each prolonged period of time, such as the location of the individual (office, at home, etc.), time of day, time in the week, etc.

Furthermore, by aggregating the data for all tested individuals, structural information and trends can be established for tasks, and for certain parameters thereof in the additional situational information (or in other context information), which can then again be used in implementing policies to boost wellbeing and/or efficiency. As mentioned, if the data shows that individuals show substantially more negative values for the third set of KPIs under a certain choice in a parameter than for another choice in said parameter, a company policy can be instituted on this, pushing (or even forcing) employees towards the more positive choice in the parameter. This can be for example, the data showing that a required admin task generates ‘bad’ (third set of) KPI values when done while working at home. If so, company policy can for instance require the required admin task to be performed while at the office.

By looking at data in greater groups of individuals (or even in subgroups), trends can be seen and acted on, thus optimizing workflow in terms of mental health, which often also improves performance.

In a preferred embodiment, at least one of the predictive models is pretrained with supplemented biometric data, said supplemented biometric data comprising the eye tracking data, the voice analysis data and the facial expression analysis data and one or more of heart rate data, skin conductance data and/or brainwave data from one or more of the individuals. This information can be provided occasionally or in a singular instance, to provide a more reliable baseline for individuals, especially when compared to the other biometric data at said time.

In a further preferred embodiment, the supplemented biometric data is processed according to step c. to a reduced feature set, and wherein the at least one of the predictive models is pretrained with the reduced feature set.

In a preferred embodiment, anomalous data and/or features are automatedly removed and/or automatedly adapted from the biometric data based on the behavioral data, the extracted feature set or the reduced feature set.

In a preferred embodiment, the biometric data is supplemented with a time stamp, allowing anomaly detection to be performed more efficiently, while also allowing separate data streams to be processed in parallel, and taken into account the other data sets at the time, giving more insight in the readings.

### EXAMPLE

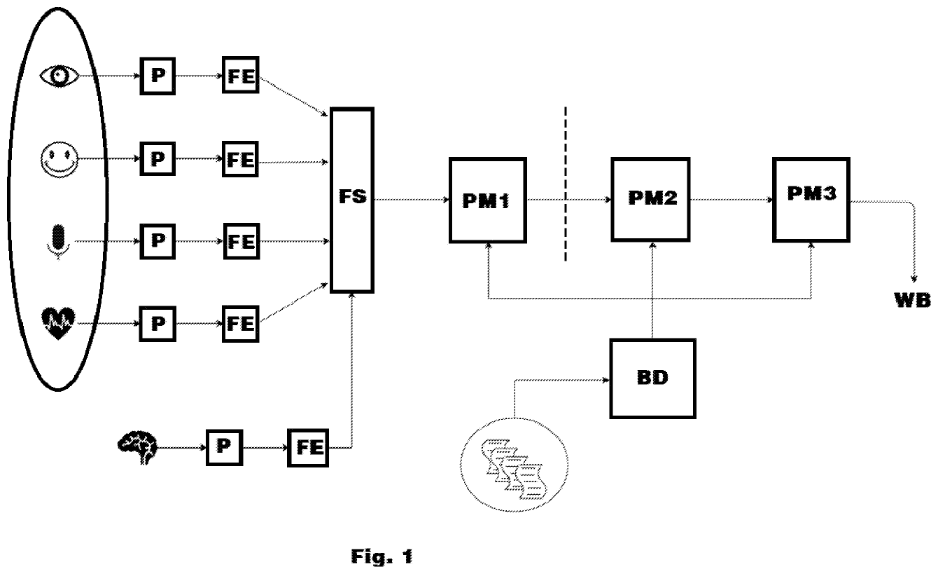

FIG. 1 shows a process flow for a possible embodiment of the invention, supplemented with a number of optional techniques. On the left side, four different types of biometric data are shown, a number of which that can be acquired via audio-visual information (first three, being eye tracking data, facial expression analysis data and voice analysis data), with the fourth being an optional data stream that provides heart rate/heartbeat information. Each type of biometric data above is acquired at the individual's working station. A further optional stream is shown at the bottom, namely brainwave data (for instance acquired via ECG), which may or may not be acquired live and/or in loco.

Each data set is preferably pre-processed (step P) and subsequently undergoes feature extraction (FE). The resulting features are then combined, and undergo feature selection (FS), resulting in a feature set that is fed into a first predictive model (PM1), which generates values for a first set of KPIs at the working station of the individual. This is then sent from the working station to a remote server (with the dotted line showing the separation between local and remote), where the second predictive model (PM2) generates values for the second set of KPIs, and feeds these to a third predictive model (PM3) which is finally converted into a wellbeing score (WB). The predictive models (PM1, PM2 and PM3) are furthermore fed with behavioral data (BD), extracted by such means as questionnaires, typically periodically.

The present invention is in no way limited to the embodiments described in the examples and/or shown in the figures. On the contrary, methods according to the present invention may be realized in many different ways without departing from the scope of the invention. For instance, the present invention was described with particular references to detection of depression and burn-out, but it is clear that the invention is likewise applicable for het detection of for instance anxiety disorders, trauma and other mental health issues.

1. A computer-implemented method for assessing and processing, preferably real-time, biometric parameters of individuals and pre-emptively detecting mental health issues, preferably burn-out and/or depression, the method comprising:
a. Acquiring audio-visual information via an electronic device provided with an image sensor from each of the individuals during work operations of each of the individuals over prolonged periods of time, said audio-visual information comprising video and/or images and associated audio output;
b. Acquiring biometric data for each of the individuals from said audio-visual information, wherein said audio-visual information is processed into the biometric data by a processor in said electronic device, said biometric data comprising at least:
i. eye tracking data, comprising at least eye position and eye movement for each of the individuals, acquired from video and/or images;
ii. facial expression analysis data, acquired from said video and/or images;
iii. voice analysis data, acquired from said audio associated output;
c. Processing said biometric data for each of the individuals, said processing comprising the following substeps, wherein the biometric data is processed by the processor in said electronic device:
i. Performing a feature extraction algorithm on the biometric data, resulting in an extracted feature set;
ii. Performing a feature selection algorithm on the extracted feature set, resulting in a reduced feature set;
d. Feeding the reduced feature set for said biometric data, preferably after step ii. of the processing step and a behavioral data set to a first predictive model, said behavioral data set comprising data associated to each of the individuals and comprising at least physical health information and/or mental health information, said first predictive model, preferably using a first machine learning model, and said first predictive model defining values for a predefined first set of key performance indicators (KPIs) from the features of the reduced feature set, wherein said first predictive model is executed on the electronic device;
e. Sending the first set of KPIs to a remote server, preferably a cloud server;
f. Feeding the first set of KPIs, and optionally the behavioral data set to a second predictive model at said remote server, thereby defining values for a predefined second set of KPIs, said second predictive model preferably using a second machine learning model, wherein said second predictive model is executed at the remote server;
g. Feeding the second set of KPIs, and optionally the behavioral data set to a third predictive model at said remote server, thereby defining values for a predefined third set of KPIs, said third predictive model preferably using a third machine learning model, wherein said third predictive model is executed at the remote server, said third set of KPIs preferably relating to a single KPI;
h. Determining a mental health assessment for each of the individuals separately based on said values of said third set of KPIS.

2. The computer-implemented method according to claim 1, wherein processing the biometric data comprises a substep of pre-processing the biometric data, said pre-processing at least comprising denoising of at least part of the biometric data, preferably at least the audio output, said step of pre-processing the biometric data preceding the substep of performing a feature extraction algorithm on the biometric data.

3. The computer-implemented method according to claim 2, wherein the steps of pre-processing and feature extraction of the biometric data is performed per set of eye tracking date, facial expression analysis data and voice analysis data separately for each of said sets.

4. The computer-implemented method according to claim 1, wherein the step of processing the biometric data comprises a substep of automatedly detecting anomalous data and/or features in the biometric data, in the extracted feature set and/or in the reduced feature set, and subsequently automatedly removing and/or automatedly adapting anomalous data and/or features therefrom.

5. The computer-implemented method according to claim 1, wherein at least two, and preferably each, of the first, second and third predictive models are different predictive models.

6. The computer-implemented method according to claim 1, wherein the first, second and/or third predictive models are machine learning models selected from the following list: Random forest, Support Vector Machine (SVM), Relevance Vector Machine (RVM), Perceptron, Artificial Neural Network (ANN), K-Means Clustering, k-Nearest Neighbours (k-NN).

7. The computer-implemented method according to claim 1, wherein the biometric data is collected through a webcam at a working station of each of the individuals.

8. The computer-implemented method according to claim 1, wherein the prolonged periods of time span at least 5 minutes, preferably at least 10 minutes, more preferably at least 30 minutes, wherein preferably multiple sets of audio-visual information are acquired over said prolonged periods of time for each of the individuals.

9. The computer-implemented method according to claim 1, wherein said individuals are divided in groups based on work content of the individuals, wherein group average comparison values are determined by averaging the values of the KPIs of the third set of KPIs and/or averaging the values of the KPIs of the second set of KPIs for each of the individuals in a group, and wherein the step of determining the mental health assessment for each of the individuals separately further takes into account the group average comparison values for the group to which the individual belongs.

10. The computer-implemented method according to claim 1, wherein the predictive models are furthermore fed with situational data associated to each of the individuals, said situation data comprising at least a geographical denomination to the work environment of the individual.

11. The computer-implemented method according to claim 1, wherein the biometric data is supplemented with a task description for the work operation of the individual during the prolonged period of time, said task description preferably selected from a predefined list of task descriptions, preferably by the individual.

12. The computer-implemented method according to claim 1, wherein at least one of the predictive models is pretrained with supplemented biometric data, said supplemented biometric data comprising the eye tracking data, the voice analysis data and the facial expression analysis data and one or more of heart rate data, skin conductance data and/or brainwave data from one or more of the individuals.

13. The computer-implemented method according to claim 12, wherein the supplemented biometric data is processed according to step c. to a reduced feature set, and wherein the at least one of the predictive models is pretrained with the reduced feature set.

14. The computer-implemented method according to claim 1, wherein based on the behavioral data, anomalous data and/or features are automatedly removed and/or automatedly adapted from the biometric data, the extracted feature set or the reduced feature set.

15. The computer-implemented method according to claim 1, wherein the biometric data is supplemented with a time stamp.
